(19) United States
(12) Patent Application Publication
(10) Pub. No.: US 2025/0073078 A1
(43) Pub. Date: Mar. 6, 2025

(54) SYSTEM AND METHOD FOR PERFORMING OPHTHALMIC LASER ASSISTED SURGERY WITH IMMERSION LIQUIDS

(71) Applicant: ALCON INC., Fribourg (CH)

(72) Inventors: Zsolt BOR, San Clemente, CA (US); Alireza Malek Tabrizi, Fremont, CA (US); Keith Watanabe, Irvine, CA (US)

(21) Appl. No.: 18/812,462

(22) Filed: Aug. 22, 2024

Related U.S. Application Data
(60) Provisional application No. 63/579,869, filed on Aug. 31, 2023.

(51) Int. Cl. A61F 9/008

(52) U.S. Cl. CPC A61F 9/00812; A61F 2009/00842; A61F 2009/00872; A61F 2009/00887

(57) ABSTRACT

Disclosed herein is a method of performing ophthalmic surgery. The surgery includes positioning a patient interface relative to a patient's eye with the patient interface at least partially defining an interface chamber with the patient's eye. The interface chamber is filled with an immersion fluid. A fluid in an anterior chamber of the patient's eye is replaced with the immersion fluid, wherein the immersion fluid matches the refractive index of the cornea. A laser system is positioned relative to the patient interface. The laser system includes a laser source configured to generate a femtosecond laser beam and an optical delivery and scanner system in communication with the laser source to direct 3D scanned and focused laser beams through the patient interface and into the patient's eye.

### TECHNICAL FIELD

The present disclosure relates to ophthalmic surgeries and related systems and methods for performing laser assisted ophthalmic surgeries.

### BACKGROUND

Ophthalmic procedures and the systems that perform them contribute to improving the quality of life of a patient by enhancing the patient's vision. Ophthalmic systems may include surgical lasers for performing an anterior segment eye procedure, such as a cataract procedure. These systems often include patient interfaces for engaging and immobilizing the patient's eye during an ophthalmic procedure. Fixating the patient's eye during the ophthalmic procedure ensures the precision and success of the ophthalmic procedure. However, patient interfaces often cause corneal wrinkles impairing the optical quality of the surgical laser beam.

### SUMMARY

Disclosed herein is a method of performing ophthalmic surgery. The surgery includes positioning a patient interface relative to a patient's eye with the patient interface at least partially defining an interface chamber with the patient's eye. The interface chamber is filled with an immersion fluid. A fluid in an anterior chamber of the patient's eye is replaced with the immersion fluid, wherein the immersion fluid includes a refractive index that matches a refractive index of a cornea of the patient's eye. A laser system is positioned relative to the patient interface. The laser system includes a laser source configured to generate a femtosecond laser beam and an optical delivery and scanner system in communication with the laser source to direct 3D scanned and focused laser beams through the patient interface and into the patient's eye.

In one embodiment, the refractive index of the immersion fluid is greater than or equal to 1.37 and less than or equal to 1.40. The patient interface includes a suction ring for engaging the eye and removing possible air bubbles in the immersion fluid. The patient interface includes a liquid interface housing extending from the suction ring and an entry window that at least partially defines the interface chamber with the liquid interface housing.

In some embodiments, the patient interface is positioned relative to the patient's eye by applying negative pressure to a suction ring on the patient interface. The immersion fluid may include Densiron-68, HWS-46-3000, or Oxane H.

In one embodiment, the ophthalmic surgery includes femtosecond laser assisted cataract surgery (FLACS) and includes replacing the fluid in the anterior chamber of the patient's eye with the immersion fluid occurs through an opening in a cornea.

In one embodiment, the ophthalmic surgery includes a femtosecond laser adjusted intraocular lens (IOL) procedure.

In one embodiment, the ophthalmic surgery includes at least one of femtosecond retinal treatment procedure, epiretinal membrane and internal limiting membrane surgery, disintegration of retinal drusens, or cutting vitreous traction fibers.

In one embodiment, the ophthalmic surgery includes a femtosecond laser-based floater removal procedure.

In one embodiment, the ophthalmic surgery includes a laser induced refractive index change (LIRIC) procedures or Perfect Lens treatment.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### BRIEF DESCRIPTION OF THE DRAWINGS

FIG. 1 illustrates a schematic diagram of a laser system positioned relative to a patient's eye.

FIG. 2 illustrates an enlarged cross-section of a liquid patient interface positioned relative to the patient's eye according to the prior art.

FIG. 3 illustrates an enlarged cross-sectional view of an immersion liquid patient interface positioned relative to the patient's eye.

FIG. 4 illustrates a method of performing ophthalmic surgery.

FIG. 5 illustrates another example method of performing a femtosecond laser assisted cataract surgery.

The foregoing and other features of the present disclosure are more fully apparent from the following description and appended claims, taken in conjunction with the accompanying drawings.

### DETAILED DESCRIPTION

Embodiments of the present disclosure are described herein. It is to be understood, however, that the disclosed embodiments are merely examples and other embodiments can take various and alternative forms. The figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

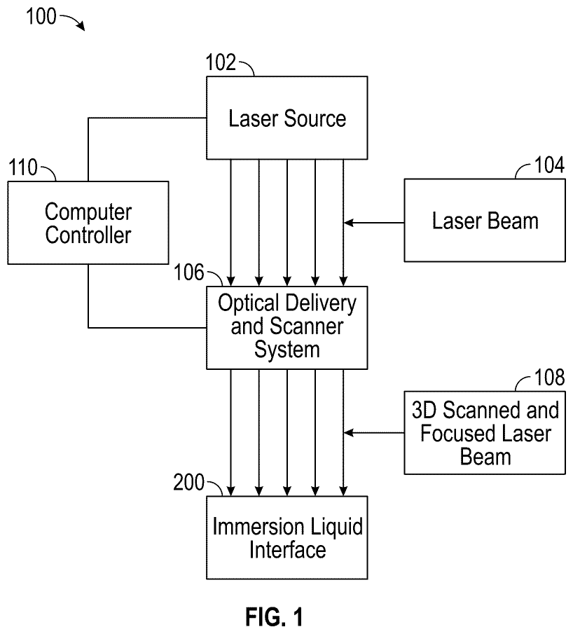

FIG. 1 illustrates a schematic diagram of a laser system 100, such as a pulsed laser system, positioned relative to an eye 20. The laser system 100 may be a separate surgical tool, or part of a larger eye surgery system, which may include other laser systems, patient or eye positioning systems, viewing systems, or any combinations thereof. In particular, the laser system 100 may be part of a surgical suite designed to provide substantially all computer-assisted devices for performing a given eye surgery.

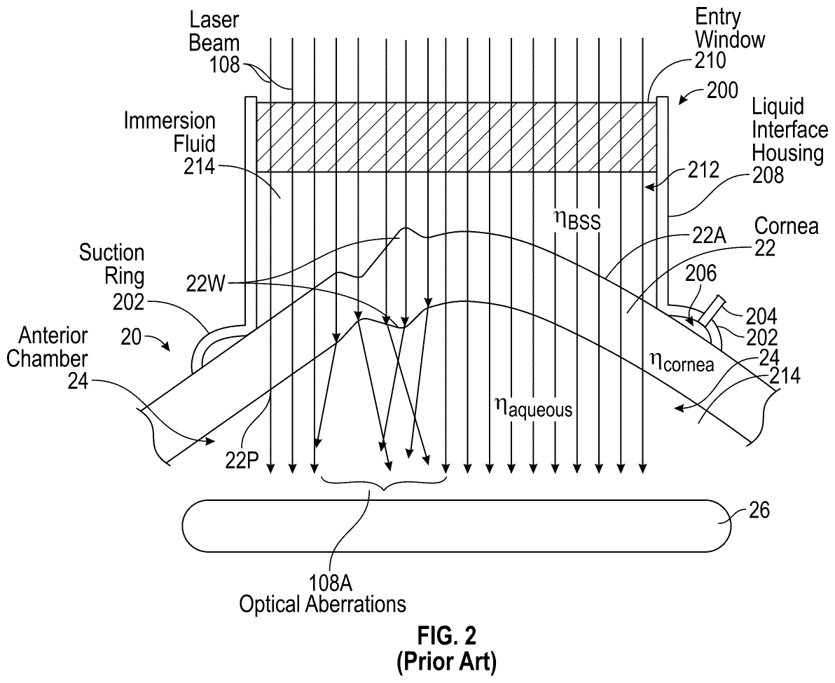

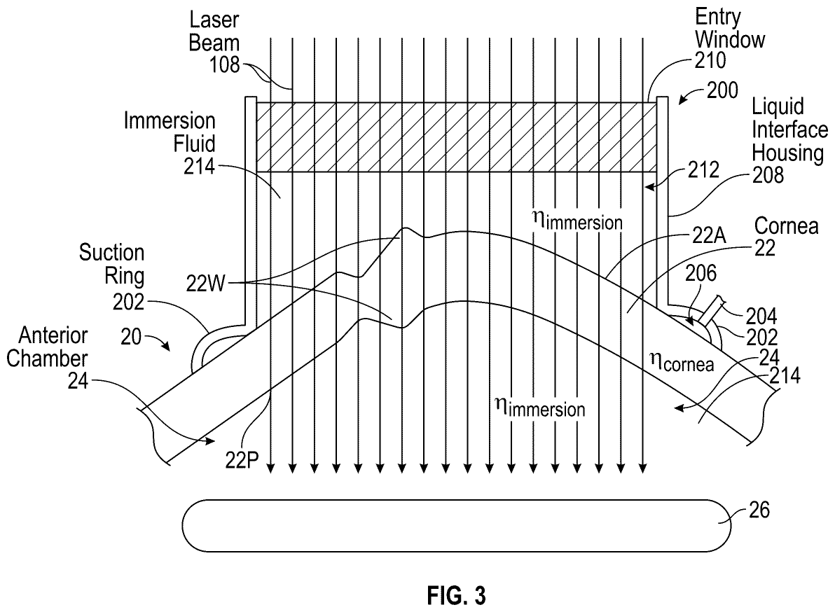

The laser system 100 includes a laser source 102, which generates laser radiation, such as a laser beam 104. The laser beam 104 may include laser radiations from a femtosecond laser. The laser system 100 can include an optical delivery and scanner system 106 for controlling 3D scanned and focused laser beams 108 during surgery in the patient's eye 20 (FIGS. 2-3). The system 106 provides transverse control axis (X- and Y-axes), longitudinal control axis (Z-axis) of 3D scanned and focused laser beams 108. “Transverse” refers to a direction at a right angle to the propagation direction of laser beam 104. “Longitudinal” refers to the propagation direction of the laser beam 104. The system 106 may be 3D scanner.

Although the laser system 100 in FIG. 1 does not show various other radiation control components, the system 106 may control 3D scanned and focused laser beams 108 in a longitudinal direction using a longitudinal control element. For example, longitudinal control element may include a longitudinally adjustable lens. Alternatively, a longitudinal control element may include a variable refractive power lens. Also, alternatively, longitudinal control element may include a deformable mirror. Further, the system 106 may contain more than one transverse control element, more than one longitudinal control element, or more than one of both. In addition, the transverse control element and the longitudinal control element may be separate devices. Although system 106 shown in FIG. 1 is depicted as one component, such a configuration is merely provided for illustrative purposes. The embodiments of the present disclosure may be configured to include multiple scanners (a system of scanners) to allow for more precise control of the 3D scanned and focused laser beams 108.

The laser source 102 and system 106 are controlled by a computer controller 110. For example, the computer controller 110 may control which wavelength of laser beam 104 is generated from the laser source 102. For instance, the computer controller 110 may configure the laser source 102 to generate a femtosecond laser 104. Additionally, the computer controller 110 may control the system 106 to change movements of the 3D scanned and focused laser beams 108.

The computer controller 110 includes at least a processing resource able to execute code to generate instructions to control operation of the laser 102. The computer controller 110 may be in physical or wireless communication with laser source 102 and system 106. The computer controller 110 may further include a memory, particularly a memory for storing instructions for the processing resource, a communications module for communicating with laser source 102 and system 106, and other components.

For simplicity, not all potential components of the pulsed laser system 100 are illustrated in FIG. 1. For example, the laser system 100 may include various components for directing, focusing, or otherwise manipulating laser beams, such as scanners, mirrors, beam expanders, or lenses. The laser system 100 may further include housings and other equipment to protect and position its components as well as patient-interface peripherals, which may be disposable.

FIGS. 2-3 illustrate the propagation of the scanned and focused laser beam through the liquid interface with and without refractive index matched immersion. For clarity, the illustration is based on parallel laser beam 108. A skilled person understands how the immersion works in case of focused laser beam 108.

FIGS. 2 and 3 illustrate the patient interface 200 positioned relative to the eye 20 in more detail. The patient interface 200 includes a suction ring 202 that fits against an outer surface of the eye 20, e.g., with a suction portion. The suction portion can be coupled to a vacuum or suction supply 204 to apply vacuum or negative pressure to expel air from a suction chamber 206, to fix the patient interface 200 relative to the eye 20. A liquid interface housing 208 extends outward from the suction ring 202 relative to the eye 20. In the illustrated example, the liquid interface housing 208 is sealed relative to the suction ring 202 and includes a cylindrical profile. The liquid interface housing 208 may be a single integral piece with the suction ring 202 or the liquid interface housing 208 could be attached to the housing.

An entry window 210 is located within the interface housing 208 and is spaced from the suction ring 202. In one example, the entry window 210 is removably attached to the interface housing 208 to allow for the insertion of an immersion fluid 214 within an interface chamber 212. The entry window 210 is made of a transparent material that allows the scanned and focused laser beams 108 to pass through the entry window 210 and into the eye 20. In the illustrated example, the interface chamber 212 is at least partially defined by a portion of the patient's eye 20, the suction ring 202, the interface housing 208, and the entry window 210.

The anterior chamber 24 is normally filled with aqueous humor as shown in FIG. 2. However, in this disclosure, the aqueous humor in the anterior chamber 24 is replaced with the immersion fluid 214 that is also located in the interface chamber 212 as shown in FIG. 3.

A refractive index of the immersion fluid 214 closely matches the refractive index of the patient's cornea 22. Such interface which is the subject of this disclosure will be called as Immersion Liquid Filled Patient Interface (ILFPI). In one example of a closely matched retractive index, a refractive index of the immersion fluid 214 is greater than or equal to 1.36 and less than or equal to 1.40. In another example, the refractive index of the immersion fluid 214 is greater than or equal to 1.37 and less than or equal to 1.40. Example immersion fluids 214 could include Densiron-68, HWS-46-3000, or Oxane H. However, other immersion fluids 214 can be used that closely match the refractive index of the cornea 22.

When the patient interface 200 is attached to the eye 20, the applanation of force from the suction ring 202 can cause wrinkles 22W to form in the cornea 22. The wrinkles 22W typically have different shapes on the anterior side 22A and posterior side 22P of the cornea 22.

With the presently used procedures illustrated in FIG. 2, the interface chamber 212 would be filled either with a balanced salt solution (BSS) or even possible water. The refractive index η of aqueous humor, water, or BSS is 1.336. The refractive index η of the cornea 22 is 1.376. Therefore, the difference in refractive index η between the cornea 22 and the BSS, aqueous humor, or water is Δη=0.04.

Let us assume that the wrinkles 22W cause a δ=5 μm thickness variation of the cornea 22. These variations would be difficult if not impossible to detect even with optical coherence tomography (OCT). Therefore, the surgeons cannot have a knowledge about the existence of the wrinkles 22W. The wrinkles 22W can cause optical aberration with a phase error Δφ of the laser beam 104 when the scanned and focused beams 108 pass through the cornea 22 into either the BSS fluid, aqueous humor, or water. EQ. 1 below is used to calculate the phase error of the laser beam 104.

$$Δϕ = 2 * π \times δ \times Δη / λ \qquad EQ . l$$

Assuming a laser wavelength of the laser beam to be λ=1.035 μm, which is the typical wavelength of a femtosecond laser, the phase error (Eq.1.) turns to be Δφ=2*p*d*D η/1=2*p*5 mm*0.04/1.035 mm=1.214 radian. The phase error caused aberrations, which increases the focal spot area and therefore reduces the intensity of the scanned and focused laser beams 108 in the focal point compared to the intensity of the un-aberrated beam by a factor of the Strehl Ratio S. The Strehl Ratio can be calculated as shown in EQ. 2 below

$$S = exp - {( Δ Φ )}^{2} \qquad EQ . 2$$

Using the two above equations, the optical aberration caused by the 5-μm wrinkle 22W with BSS, aqueous humor, or water reduces the intensity of the scanned and focused laser beams 108 in the focal point of the optical delivery system 106 from 100% to 22.9% as shown in EQ. 3 below.

$$S = exp - {DF}^{2} = exp - {( 1.214 )}^{2} = 0.229 = 22.9 \% \qquad EQ . 3$$

During surgery or other treatments, the reduction of intensity can be compensated for by using higher laser pulse energies by about 1/S times but increasing the laser pulse energy might have clinical disadvantages. Controlling the wrinkle 22W elevation d in the cornea 22 on this scale is practically impossible. Also, the wrinkles 22W differ from patient to patient and also can be differ from docking to docking on the same patient. The wrinkles 22W are also caused and are changing during the laser treatment due to minimal lateral movements of the patient's head or eye 20.

However, by using immersion liquids in the anterior chamber 24 of the eye 20 and in the interface chamber 212 of the ILFPI 208, the Strehl ratio can be considerably improved. Table 1 below which shows the calculated Strehl ratio for different immersion liquids and assuming the same 1.035 mm laser wavelength and d===5 mm corneal thickness error for clinically available ophthalmic tapenades.

TABLE 1

| | Densiron-68 | Oxane H | HWS 46-3000 | BSS/aqueous |
|---|---|---|---|---|
| Refractive | 1.39 | 1.4 | 1.37 | 1.336 |
| index | | | | |
| Refractive | 0.014 | 0.024 | 0.006 | 0.04 |
| index | | | | |
| difference | | | | |
| DF | 0.0425 | 0.728 | 0.182 | 1.214 |
| Calculated | 0.835 | 0.587 | 0.967 | 0.229 |
| Strehl ratio | | | | |

As shown in Table 1 above, by decreasing the refractive index difference Dh the Strehl ratio is approaching a maximal possible Strehl ratio of 1. For example, by filling the anterior chamber 24 and the interface chamber 212 of the ILFPI 208 with HWS 46-3000 which has a refractive index of 1.37, the Strehl Ratio is 0.967. This is to be compared to the S=0.229 Strehl ratio when using BSS or aqueous. This illustrates how using the immersion liquid HWS 46-3000 increases the intensity in the focus by a factor of 0.967/0.229=4.24 times.

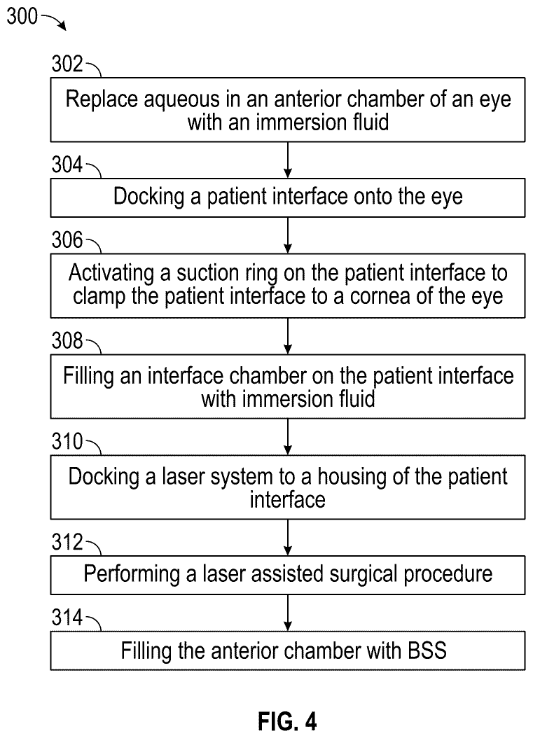

FIG. 4 illustrates a method 300 of performing laser assisted ophthalmic surgery that reduces the formation of optical aberrations 108A. The method 300 includes replacing the aqueous humor in the anterior chamber 24 of eye 20 with an immersion fluid 214, such as one of the immersion fluids identified above, that closely matches the refractive index of the cornea (Block 302). The exchange can occur with a syringe or through an opening in the cornea 22. In some embodiments, an immersion fluid that matches, or even closely matches the refractive index of the cornea may include the immersion fluid including a refractive index that is within a threshold value of the refractive index of the cornea. The threshold may include a refractive index difference of less than 0.04, less than 0.03, less than 0.02, less than 0.01, between 0.04 and 0.00, between 0.03 and 0.010, or any other ranges using any of the preceding values as end points.

The patient interface 200 is docked relative to the patient's eye 20 (Block 304). A vacuum source 204 is activated such that the suction ring 202 on the patient interface 200 clamps to the cornea 22 of the patient's eye 20 (Block 306). The patient interface 200 is configured to form the interface chamber 212 at least partially with the patient's eye 20 for filling with the immersion fluid 214 adjacent the cornea 22 (Block 308).

The laser system 100 is docked relative to the patient interface 200 (Block 310) for performing a laser assisted surgical procedure (Block 312). In the illustrated example, the laser system 100 includes a laser source configured to generate a femtosecond laser beam and a focusing lens in optical communication with the laser source to direct the laser beam through the patient interface 200 and into the patient's eye 20.

An example surgical procedure for utilizing the method 300 includes a femtosecond laser adjusted intraocular lens (IOL) procedure, such as laser-induced refractive index change (LIRIC) or Perfect Lens treatment. One feature of utilizing an IOL, such as a light adjustable lens (LAL), is the ability to refine the refractive properties of the lens after it has been surgically implanted in the eye 20. In particular, the refractive properties of the LAL are adjustable by exposing the lens to the femtosecond laser. This allows the refractive properties of the LAL to be adjusted once the lens has been implanted for a period of time to provide further improved vision. One feature of the reduction in optical aberrations 108A of the laser from the method 300 is an improvement in the precision of the laser beams 104 when adjusting the refractive properties of the LAL. This results in further improved vision for the patient.

Another example surgical procedure for utilizing the method 300 includes retinal microsurgery using femtosecond laser pulses. The retinal microsurgery can include cutting the vitreous traction fibers anchored to the retina. The traction fibers can tear off the retina upon age related vitreous detachment.

Another example surgical procedures for utilizing the method 300 includes epiretinal membrane and internal limiting membrane surgery or optogenetic treatment of the retina to cure AMD (Agerelated Macular Degeneration), retinitis pigmentosa and other retinal diseases.

Yet another example surgical procedure for utilizing the method 300 includes disintegration of retinal drusens. Drusens are lipid and protein deposits developing in the retina in the early phase of AMD.

Yet another example ophthalmic surgical procedure for utilizing the method 300 includes femtosecond laser-based removal of vitreous floaters. The floaters include clumps of collagen proteins that form in the vitreous casting moving shadows onto the retina. In ophthalmic laser surgery, a surgeon may direct a laser beam into the vitreous of the eye 20 to treat eye floaters. Eye floaters are clumps of collagen proteins that form in the vitreous. These clumps can disturb vision with moving shadows and distortions. The laser beam may be used to fragment the floaters to improve vision. The method 300 improves the precision of the laser beams targeted at the eye floaters to improve fragmentation.

Once the surgical procedure is completed in Block 312, the immersion fluid in the anterior chamber 24 is replaced with BSS (Block 314).

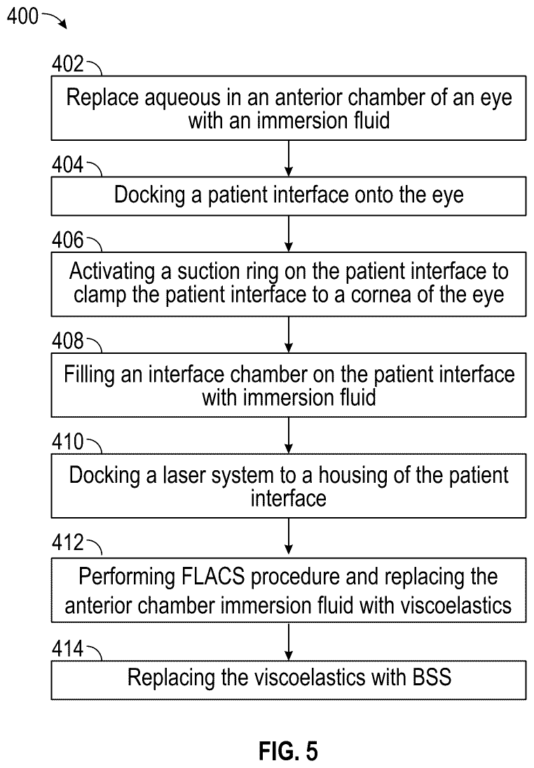

FIG. 5 illustrates a method 400 of performing a femtosecond laser assisted cataract surgery (FLACS) that reduces the formation of optical aberrations 108A. The method 400 includes replacing the aqueous humor in the anterior chamber 24 of eye 20 with an immersion fluid 214, such as one of the immersion fluids identified above, that closely matches the refractive index of the cornea (Block 402). The exchange can occur with a syringe or through an opening in the cornea 22.

The patient interface 200 is docked relative to the patient's eye 20 (Block 404). A vacuum source 204 is activated such that the suction ring 202 on the patient interface 200 clamps to the cornea 22 of the patient's eye 20 (Block 406). The patient interface 200 is configured to form the interface chamber 212 at least partially with the patient's eye 20 for filling with the immersion fluid 214 adjacent the cornea 22 (Block 408).

The laser system 100 is docked relative to the patient interface 200 (Block 410) for performing the FLACS procedure (Block 412). In the illustrated example, the laser system 100 includes a laser source configured to generate a femtosecond laser beam and a focusing lens in optical communication with the laser source to direct the laser beam through the patient interface 200 and into the patient's eye 20. The FLACS procedure includes, laser capsulorhexis, lens fragmentation using the femtosecond laser, filling the anterior chamber 24 with viscoelastics (i.e., replacing the immersion liquid 214 in the anterior chamber 24 with the viscoelastics), removing the circumcised capsule, phacoemulsification, removal of lens debris, cleaning the capsule, and implanting the IOL in place of the lens 26. The viscoelastics in the anterior chamber 24 are then replaced with BSS at Block 414.

The surgical procedures identified above for the methods 300 and 400 are based on four photon laser tissue interaction. Four photon interaction means that the yield of the processes is proportional to the fourth exponent of the laser intensity in the focal point i.e., to fourth exponent of the Strehl ratio. If the BSS is used in the anterior chamber 24 and in the immersion chamber 214 of the patient interface 200 the Strehl ratio is 0.229. This results in the yield of the four photon initial steps being 0.229⁴=0.00275 times less than for S=1 error-free optical delivery system and 0.967⁴=0.87 for the ILFPI using HWS 46-3000 immersion liquid. These numerical examples clearly demonstrate the importance of the well-matched immersion liquid interfaces.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed:

1. A method of performing ophthalmic surgery, comprising:
positioning a patient interface relative to a patient's eye, wherein the patient interface at least partially defines an interface chamber with the patient's eye;
filling the interface chamber with an immersion fluid;
replacing a fluid in an anterior chamber of the patient's eye with the immersion fluid, wherein the immersion fluid includes a refractive index that matches a refractive index of a cornea of the patient's eye; and
positioning a laser system relative to the patient interface, the laser system including:
a laser source configured to generate a femtosecond laser beam; and
an optical delivery and scanner system in communication with the laser source to direct 3D scanned and focused laser beams through the patient interface and into the patient's eye.

2. The method of claim 1, wherein the refractive index of the immersion fluid is greater than or equal to 1.37 and less than or equal to 1.40.

3. The method of claim 1, wherein the patient interface includes a suction ring for engaging the patient's eye and removing possible air bubbles in the immersion fluid.

4. The method of claim 3, wherein the patient interface includes a liquid interface housing extending from the suction ring.

5. The method of claim 4, wherein the patient interface includes an entry window at least partially defining the interface chamber with the liquid interface housing.

6. The method of claim 1, wherein positioning the patient interface relative to the patient's eye includes applying negative pressure to a suction ring on the patient interface.

7. The method of claim 1, wherein the immersion fluid includes Densiron-68.

8. The method of claim 1, wherein the immersion fluid includes HWS-46-3000.

9. The method of claim 1, wherein the immersion fluid includes Oxane H.

10. The method of claim 1, wherein the ophthalmic surgery includes femtosecond laser assisted cataract surgery (FLACS).

11. The method of claim 10, wherein replacing the fluid in the anterior chamber of the patient's eye with the immersion fluid occurs through an opening in a cornea.

12. The method of claim 1, wherein the ophthalmic surgery includes a femtosecond laser adjusted intraocular lens (IOL) procedure.

13. The method of claim 1, wherein the ophthalmic surgery includes at least one of femtosecond retinal treatment procedure, epiretinal membrane and internal limiting membrane surgery, disintegration of retinal drusens, or cutting vitreous traction fibers.

14. The method of claim 1, wherein the ophthalmic surgery includes a femtosecond laser-based floater removal procedure.

15. The method of claim 1, wherein the ophthalmic surgery includes a laser induced refractive index change (LIRIC) procedures or Perfect Lens treatment.

16. A system for performing ophthalmic surgery, comprising:
a patient interface for attaching to a patient's eye, wherein the patient interface at least partially defines an interface chamber;
an immersion fluid for filling the interface chamber and an anterior chamber of the eye, wherein the immersion fluid includes a refractive index of greater than or equal to 1.36 and less than or equal to 1.40; and
a laser system positionable relative to relative to the patient interface, the laser system including:
a laser source configured to generate a femtosecond laser beam; and
a focusing lens in optical communication with the laser source to direct the laser beam through the patient interface and into the patient's eye.

17. The system of claim 16, wherein the patient interface includes a suction ring for engaging the patient's eye, a liquid interface housing extending from the suction ring, and an entry window at least partially defining the interface chamber with the liquid interface housing.

18. The system of claim 17, wherein the ophthalmic surgery includes femtosecond laser assisted cataract surgery (FLACS).

19. The system of claim 16, wherein the ophthalmic surgery includes a femtosecond laser adjusted intraocular lens (IOL) procedure.

20. The system of claim 16, wherein positioning the patient interface relative to the patient's eye includes applying negative pressure to a suction ring on the patient interface.
